(19) United States
(12) Patent Application Publication
(10) Pub. No.: US 2011/0183925 A1
(43) Pub. Date: Jul. 28, 2011

(54) ANTI-INFLAMMATORY PEPTIDE

(75) Inventors: Kenji Sato, Kyoto (JP); Shin Ono, Toyama (JP); Yoshio Suzuki, Tokyo (JP)

(73) Assignee: NISSHIN PHARMA INC., Chiyoda-ku, Tokyo (JP)

(21) Appl. No.: 13/120,371

(22) PCT Filed: Sep. 22, 2008
(86) PCT No.: PCT/JP2008/067076

§ 371 Date: Mar. 22, 2011

(51) Int. Cl. A61K 38/05; A61K 38/06; C07K 5/08; C07C 229/00

(52) U.S. Cl. 514/21.9; 514/21.91; 530/331; 560/155

(57) ABSTRACT

Provided is an anti-inflammatory composition which has a high efficacy, causes no concern about side effects, is easy to ingest, and can also be administered for a long period of time because of its low cost and high safety. The present invention relates to a peptide comprising an amino acid sequence represented by pyroGlu-(X)n-A or a salt thereof, wherein X is independently Gln, Asn, or Pro; A represents Gln, Asn, Leu, Ile, Met, Val, or Phe; and n represents an integer of 0 to 2, and an anti-inflammatory composition comprising the same.

### TECHNICAL FIELD

The present invention relates to a peptide having an anti-inflammatory activity and an anti-inflammatory composition containing the peptide as an active ingredient.

### BACKGROUND ART

Tumor necrosis factor (TNF), particularly TNF-α, is known to be released from inflammatory cells and cause various cytotoxic reactions, immunological reactions and inflammatory reactions. TNF-α is known to be involved in the occurrence and prolongation of many inflammatory and autoimmune diseases and further cause serious septicemia and septic shock when it is released into the blood and acts systemically. Because TNF-α is a factor associated widely with the immune system of a living body, the development of agents inhibiting TNF-α is actively carried out. TNF-α is biosynthesized in an inactive form and becomes an active form by being cleaved by protease; the enzyme responsible for the activation is called a tumor necrosis factor-converting enzyme (TACE). Thus, a substance inhibiting this TACE can treat, improve, or prevent diseases, pathologic conditions, abnormal conditions, troubles, adverse symptoms and the like ascribed to TNF-α.

Interleukin-1 (IL-1) is the major inflammatory cytokine stimulating the production of prostaglandin, collagenase, and phospholipase, the degranulation of basophils and eosinophils, and the activation of neutrophils. IL-1 has extremely wide-ranging physiological effects. It elicits inflammatory reaction locally or systemically through the activation or promotion of differentiation/proliferation of immune cells, and takes part in pyrexia, the induction of acute phase proteins, the activation of osteoclasts, and the like. Because IL-1 is a factor associated widely with the immune system of a living body, the development of agents inhibiting IL-1 is actively carried out. IL-1 has subtypes, IL-1α and IL-1β, both of which are biosynthesized in an inactive form and becomes an active form by being cleaved by protease. The enzyme responsible for the activation of IL-1β is called caspase-1 (also known as an interleukin-1β-converting enzyme (ICE)). Thus, a substance inhibiting this ICE can treat, improve, or prevent diseases, pathologic conditions, abnormal conditions, troubles, adverse symptoms and the like ascribed to IL-1.

The prior art discloses an ingredient derived from the tree Morinda citrifolia L as a TACE inhibitor (Patent Document 1). Cbz-Val-Ala-(OMe)-fluoromethyl ketone is also known as an ICE inhibitor (Patent Document 2). However, these ingredients are not easy to obtain and, if obtainable, have problems with the ease of ingestion, safety, and the like.
Patent Document 1: JP Patent Publication (Kokai) No. 2007-016015 A
Patent Document 2: JP Patent Publication (Kokai) No. 11-302192 A (1999)

### DISCLOSURE OF THE INVENTION

An object of the present invention is to provide an anti-inflammatory composition which has a high efficacy, causes no concern about side effects, is easy to ingest, and can also be administered for a long period of time because of its low cost and high safety.

As a result of intensively searching for a substance having a tumor necrosis factor-converting enzyme (TACE)-inhibiting effect and a substance having a caspase-1 (ICE)-inhibiting effect, the present inventors have found that a peptide having a specific sequence has a TACE-inhibiting activity and an ICE-inhibiting activity, thereby accomplishing the present invention.

Thus, the present invention includes the following invention:

(1) A peptide comprising an amino acid sequence represented by the formula: pyroGlu-(X)n-A or a salt thereof, wherein X are the same or different and are each independently Gln, Asn, or Pro; A represents Gln, Asn, Leu, Ile, Met, Val, or Phe; and n represents an integer of 0 to 2;

(2) The peptide or a salt thereof according to (1), wherein X represents Gln or Pro; A is Gln, Leu, Met, Val, or Phe; and n represents 0 or 1;

(3) The peptide or a salt thereof according to (2), wherein the peptide is selected from the group consisting of pyroGlu-Leu, pyroGlu-Val, pyroGlu-Met, pyroGlu-Phe, pyroGlu-Gln-Gln, and pyroGlu-Pro-Gln;

(4) An anti-inflammatory composition comprising at least one peptide or salt thereof according to any of (1) to (3) as an active ingredient;

(5) The composition according to (4), wherein the composition is for suppressing inflammation by inhibiting a tumor necrosis factor-converting enzyme and/or caspase-1;

(6) The composition according to (4) or (5), wherein the composition is for preventing, improving, or treating an inflammatory disease or condition in which tumor necrosis factor and/or interleukin is involved; and

(7) The composition according to any of (4) to (6), wherein the composition is in the form of food.

According to the present invention, an anti-inflammatory composition is provided which has higher safety than that of treatment using a conventional pharmaceutical product and can be ingested in a simple manner.

### BEST MODE FOR CARRYING OUT THE INVENTION

Preferred embodiments of the present invention will be specifically described below.

The present inventors have found that a peptide comprising an amino acid sequence represented by pyroGlu-(X)n-A or a salt thereof (the peptide is hereinafter sometimes referred to as the peptide of the present invention) has an activity inhibiting a tumor necrosis factor-converting enzyme and/or caspase-1 and has an anti-inflammatory effect. Here, pyroGlu indicates pyroglutamic acid; X is independently Gln (glutamine), Asn (asparagine), or Pro (proline), preferably Gln or Pro; A represents Gln, Asn, Leu (leucine), Ile (isoleucine), Met (methionine), Val (valine), or Phe (phenylalanine), preferably Gln, Leu, Met, Val, or Phe; and n represents 0, 1, or 2, preferably 0 or 1. Examples of the peptide represented by the formula include pyroGlu-Leu, pyroGlu-Val, pyroGlu-Met, pyroGlu-Phe, pyroGlu-Gln-Gln, and pyroGlu-Pro-Gln.

Pyroglutamic acid is glutamic acid whose γ-position amide group and a-position amino group are cyclized. The peptide of the present invention may be partial hydrolysates of a natural or recombinant protein, a peptide prepared by a chemical synthesis method or a genetic engineering technique, or a combination thereof.

The amino acids constituting the peptide of the present invention may be those in D-form, L-form, or DL-form (racemic form); however, they are preferably those in L-form. When the peptide of the present invention is prepared by the partial hydrolysis of a natural protein, the constituent amino acids are all those in L-form. When the peptide of the present invention is prepared by a chemical synthesis method, there may be prepared a peptide whose constituent amino acids are all those in L-form or in D-form or a peptide in which any of the amino acids is that in L-form and the remaining are those in D-form; both of the peptides are encompassed within the present invention.

The composition of the peptide of the present invention can be determined by an amino acid analysis method. Since an acid hydrolysis method widely in use converts both pyroglutamic acid and glutamine to glutamic acid, a method is preferably used which involves quantifying glutamine and pyroglutamic acid after decomposition using enzymes specific therefor. When the peptide is a synthetic, the composition can be determined from the amount, proportion or the like of each amino acid used in synthesis.

The salt of the peptide of the present invention is, not particularly limited, provided that it is a salt acceptable pharmaceutically or as a food; examples thereof include an acid addition salt and a base addition salt. Examples of the acid addition salt include a salt with an inorganic acid such as hydrochloric acid, sulfuric acid, nitric acid and phosphoric acid and a salt with an organic acid such as acetic acid, malic acid, succinic acid, tartaric acid and citric acid. Examples of the base addition salt include a salt with an alkali metal such as sodium and potassium, a salt with an alkali earth metal such as calcium and magnesium, and a salt with an amine such as ammonium and triethylamine.

When the peptide of the present invention is prepared by the partial hydrolysis of a natural protein, a well-known method can be properly adopted as a method for hydrolyzing the protein. Specific examples thereof include a method involving hydrolysis using an acid and a method involving hydrolysis using a protease.

The natural protein used in the hydrolysis may be any available protein; however, it is preferably a protein whose safety has been identified. Examples of such a protein include an animal protein derived from the meat, skin, milk, blood or the like of an animal and a plant protein derived from a cereal such as rice and wheat and a fruit such as persimmon and peach. Among these, a protein such as gluten contained in a wheat seed is known to be rich in glutamine and is preferable as a raw material for preparing the peptide of the present invention.

The method for hydrolyzing a protein using an acid may adopt a conventional method. The acid may be a mineral acid such as sulfuric acid, hydrochloric acid, nitric acid, phosphric acid and sulfurous acid, an organic acid such as oxalic acid, citric acid, acetic acid and formic acid, or the like.

When the hydrolysis is carried out using an acid, the concentration of the protein in an aqueous medium needs to be properly regulated depending on the type and normality of the acid; the protein is preferably treated after typically adjusting its concentration to 1.0 to 80% by mass.

When the protein is hydrolyzed using a protease, one or more proteases may be allowed to act thereon in an aqueous medium to form hydrolysates. Preferred are a method using an acid protease alone and a method using an acid protease and a neutral protease or an alkaline protease in that the hydrolysis can be efficiently carried out. When a plant protein is used as the protein, the starch or fiber contained in the plant sometimes poses an impediment for a protease action or in purification. In such a case, a glycolytic enzyme such as amylase or cellulase is preferably allowed to act before and after causing the above-described protease to act or together with the protease.

Methods for purifying the protein hydrolysates thus obtained include a method involving filtering insoluble matter, a method involving performing fractionation (extraction) using a water-containing alcohol or the like, and a method involving purification by gel filtration chromatography, high-performance liquid chromatography (HPLC), or autofocusing.

When the peptide of the present invention is prepared by a chemical synthesis method, any of a liquid-phase synthesis method and a solid-phase synthesis method may be used. Preferred is a solid-phase synthesis method which involves fixing the C-terminus of an amino acid or a peptide to a solid-phase support via a linker and successively extending amino acids towards the N-terminus. When the solid-phase synthesis method is adopted, a peptide synthesizer (e.g., PSSM8 from Shimadzu or Model 433A from ABI) can also be used for synthesis.

The solid-phase support used for solid-phase synthesis may be any solid-phase support having the property of binding to the carboxyl group of Gln, Asn, Leu, Ile, Met, Val or Phe as the C-terminal amino acid of the peptide of the present invention; examples thereof include benzhydrylamine resin (BHA resin), chloromethyl resin, oxymethyl resin, aminomethyl resin, methylbenzhydryl resin (MBHA resin), acetamidomethyl resin (PAM resin), p-alkoxybenzylalcohol resins (Wang resins), 4-aminomethylphenoxymethyl resin, and 4-hydroxymethylphenoxymethyl resin.

As a specific example of synthesis, a protocol for preparing pyroGlu-Gln-Gln as the peptide of the present invention will be shown below.

Glutamine (Gln) as the C-terminal amino acid, in which the carboxyl group is protected is provided, and glutamine (Gln) as the second amino acid, in which the amino group is protected with a protective group such as a Boc (tert-butyloxycarbonyl) group or a Fmoc (9-fluorenylmethoxycarbonyl) group and the carboxyl group is activated, is subsequently condensed therewith. The protective group of the amino group of the N-terminal glutamine is then removed from the resulting Gln-Gln dipeptide, and glutamine (Gln) as the third amino acid, in which the amino group is protected with a protective group such as a Boc (tert-butyloxycarbonyl) group or a Fmoc (9-fluorenylmethoxycarbonyl) group and the carboxyl group is activated, is subsequently condensed therewith. When the solid-phase synthesis method is used, the carboxyl group of the C-terminal glutamine needs only bind to the solid-phase support instead of protecting the carboxyl group.

The carboxyl group activation can be carried out by causing the carboxyl group to react with any of various reagents to form a corresponding acid chloride, acid anhydride or mixed acid anhydride, an azide, or an active ester such as -ONp or -OBt. The peptide condensation reaction may also be performed in the presence of a condensing agent and a racemization inhibitor such as a carbodiimide reagent (e.g., dicyclohexylcarbodiimide (DCC), water-soluble carbodiimide (WSCD), or carbodiimidazole), tetraethyl pyrophosphate, or 1-hydroxybenzotriazole (HOBt).

After the end of synthetic reaction, for the solid-phase synthesis method, the resultant peptide can be dissociated from the solid-phase support with all protective groups removed, followed by washing to provide a tripeptide, Gln-Gln-Gln, in the form of a crude peptide. Subsequently, glutamine at the N-terminus can be converted to pyroglutamic acid through cyclization to provide the peptide of the present invention. The cyclization gradually proceeds in an aqueous solution; however, its speed can be accelerated by raising the temperature. The peptide can also be prepared by subjecting pyroglutamic acid as the N-terminal amino acid to the condensation reaction.

When the liquid-phase synthesis method is used, the peptide can be synthesized in the same manner as that for the solid-phase synthesis method with the exception that the C-terminal amino acid is not bound to the solid-phase support. The thus-obtained crude peptide containing the peptide of the present invention can be obtained as a highly purified peptide by proper purification using a well-known method such as high-performance liquid chromatography (HPLC).

As described above, for the chemical synthesis method for peptide, amino acids can be successively subjected to condensation and extension from the C-terminus toward the N-terminus to synthesize the peptide of the present invention having the desired amino acid sequence. Here, amino acids in L- or D-form can also be used to synthesize a peptide in which any of the amino acids is that in L-form and the remaining are those in D-form.

The peptide of the present invention thus obtained has an activity inhibiting a tumor necrosis factor-converting enzyme (TACE) and/or caspase-1 (ICE).

The TACE-inhibiting activity can be measured by a method which involves causing TACE to react with inactive TNF-α and measuring the production amount and activity of the resultant TNF-α, a method which involves causing TACE with a TACE-specific substrate and measuring the amount of the resultant product, or the like. A commercially available measurement kit (from Merck) may also be used.

The ICE-inhibiting activity can be measured by a method which involves causing ICE to react with inactive IL-1β and measuring the production amount and activity of the resultant IL-1β, a method which involves causing ICE with an ICE-specific substrate and measuring the amount of the resultant product, or the like. A commercially available measurement kit (from R&D Systems) may also be used.

TACE is involved in the activation of tumor necrosis factor (TNF), particularly TNF-α, known to be released from inflammatory cells and cause various cytotoxic reactions, immunological reactions and inflammatory reactions. Thus, the peptide of the present invention having an activity inhibiting this TACE has an activity suppressing inflammation, particularly inflammation ascribed to tumor necrosis factor (preferably TNF-α). ICE is involved in the activation of interleukin, particularly IL-1β, which is the major inflammatory cytokine stimulating the production of prostaglandin, collagenase, and phospholipase, the degranulation of basophils and eosinophils, and the activation of neutrophils and elicits inflammatory reaction locally or systemically. Thus, the peptide of the present invention having an activity inhibiting this ICE has an activity suppressing inflammation, particularly inflammation ascribed to interleukin (preferably IL-1, more preferably IL-1β).

For the purpose of the present invention, inflammation is a phenomenon resulting from the immune response of a living body to injury or stimulation due to a physical, chemical or biological factor. It often causes pain, heat sensation, redness, and swelling in an inflamed tissue and further sometimes results in the functional depression or functional loss of the inflamed tissue.

Thus, the present invention also relates to an anti-inflammatory composition, particularly an anti-inflammatory composition for suppressing inflammation by inhibiting TACE and/or ICE, containing the peptide of the present invention as an active ingredient (hereinafter sometimes referred to as the composition of the present invention). The composition of the present invention can also be used as a composition for preventing, improving, or treating an inflammatory disease or condition in which tumor necrosis factor (particularly TNF-α) and/or interleukin (particularly IL-1β) is involved. The composition of the present invention may contain only one or more peptides of the present invention. The present invention also relates to a method for suppressing inflammation, particularly a method for suppressing inflammation by inhibiting TACE and/or ICE, comprising administering the peptide or composition of the present invention to a mammal. The present invention also relates to a method for preventing, improving, or treating an inflammatory disease or condition in which tumor necrosis factor (particularly TNF-α) and/or interleukin (particularly IL-1β) is involved, comprising administering the peptide or composition of the present invention to a mammal.

Specific examples of the inflammatory disease or condition, in which tumor necrosis factor and/or interleukin is involved, include arthritis, inflammation, rheumatism, inflammatory bowel disease, Crohn disease, reflux esophagitis, emphysema, asthma, chronic obstructive lung disease, Alzheimer disease, Sjogren syndrome, cachexia, pollen disease, allergic reaction, food allergy, allergic contact hypersensitivity, contact dermatitis, cancer, tissue ulcer formation, restenosis, periodontal disease, epidermolysis bullosa, osteoporosis, transplantation rejection, troubles such as implant pain, troubles such as prosthesis pain, arteriosclerosis, aortic/arterial aneurism, congestive heart failure, myocardial infarction, cerebral ischemia, ischemia reperfusion symptoms, endometriosis, systemic allergy, neurodegenerative disorder, autoimmune injury, Huntington disease, Parkinson disease, migraine, depression, osteoclastic disease, meningitis, neuropathic pain, amyotrophic lateral sclerosis, multiple sclerosis, pachyderma, psoriasis, ocular angiogenesis, conjunctival disorder, corneal disorder, corneal cicatrization, scleritis, macular degeneration, abnormal wound healing, burn, diabetes, tumor invasion, tumor proliferation, tumor metastasis, AIDS, septicemia, and septic shock. The composition of the present invention is particularly effective in the prevention, improvement and treatment of rheumatism.

Defatigation, chronic fatigue syndrome, muscular pain, and the like are known as other diseases or pathologic conditions in which tumor necrosis factor and/or interleukin is involved. The present invention is also particularly effective thereagainst.

According to the present invention, the prevention of diseases or conditions includes suppressing and delaying the occurrence of the diseases or conditions, and also includes prevention before developing the diseases or conditions as well as prevention against recurrence of the diseases or conditions after treatment. According to the present invention, the treatment of diseases or conditions includes curing the diseases or conditions, improving their symptoms, and suppressing the progression of the symptoms. The anti-inflammatory activity refers to an activity suppressing inflammation, and the suppression of inflammation encompasses the prevention and treatment of the inflammation and includes suppressing the inflammation, suppressing the progression of the inflammation, curing the inflammation, and improving the inflammation.

For the purpose of the present invention, the mammal refers to a homeotherms; examples thereof include primates such as humans and monkeys, rodents such as mice, rats and rabbits, pet animals such as dogs and cats, and domestic animals such as cattle, horses and pigs. The composition of the present invention is suitable for administration to primates, particularly humans. It is particularly preferable to administer the composition of the present invention to humans having inflammation, humans having been diagnosed as having inflammation, humans having a possibility of developing inflammation, and humans required to be prevented from inflammation.

In the usual case, the composition of the present invention is administered in the range of 0.01 to 20 g/day/adult, preferably 0.1 to 10 g/day/adult in terms of the mass of the peptide. When the peptide used in the present invention is prepared by the partial hydrolysis of a natural protein, its dosage can also be further increased since it is a peptide having high safety, derived from a natural product. The dosage is preferably increased or decreased properly while observing the efficacy thereof and the like. The daily dosage may be administered or ingested at once, but is preferably administered in several portions.

The form of the composition of the present invention is not particularly limited; for example, it may be prepared as a pharmaceutical composition or a food (including a feed).

When prepared as a pharmaceutical composition, the composition of the present invention is typically prepared as a preparation containing the peptide of the present invention and a pharmaceutically acceptable carrier. The pharmaceutically acceptable carrier generally refers to a filler, a diluent, an encapsulating material, or the like which are inactive, nontoxic, solid or liquid, and unreactive with the peptide of the present invention as an active ingredient; examples thereof include solvents or dispersive media such as water, ethanol, polyols (e.g., glycerol, propylene glycol, and liquid polyethylene glycol), suitable mixtures thereof, and vegetable oil.

The dosage form of the pharmaceutical composition is not particularly limited, and may be any dosage form including dosage forms for oral administration such as tablets, pills, granules, dust formulations, fine grains, powders, capsules, syrups, drinkable preparations, solutions, suppositories and liquid meals and dosage forms for parenteral administration such as sublingual tablets, nasal sprays, and injectable solutions.

Methods for administering the composition of the present invention include administration methods generally used for pharmaceutical administration, such as intravenous administration, intramuscular administration, and subcutaneous administration, in addition to oral administration. Administration methods which involve absorption through mucous membranes, such as rectal, sublingual and intranasal administration, other than the gastrointestinal tract can also be adopted. Here, the pharmaceutical composition can be administered in the form of, for example, a suppository, a sublingual tablet, or a nasal spray.

The content of the peptide of the present invention in the pharmaceutical composition varies depending on the form thereof; however, it is generally 0.001% to 99% by mass, preferably 0.01% to 90% by mass, more preferably 1% to 85% by mass, and still more preferably 5% to 80% by mass, on a dry basis. It is preferable that the daily dosage can be controlled so as to achieve the above-described daily ingestion dose per adult.

When the composition of the present invention is prepared as a food, the form thereof is not particularly limited. The food includes a beverage and also encompasses a health food and a functional food. The health food and the functional food can be prepared in the form of various preparations, such as tablets, pills, granules, dust formulations, fine grains, powders, capsules, syrups, drinkable preparations, solutions, and liquid meals. The food in the form of preparations can be produced in the same manner as that for the above-described pharmaceutical composition. It can be produced, for example, using a conventional means after adding a suitable excipient (e.g., starch, processed starch, lactose, glucose, or water). Specific examples of the food further include coffee beverages, tea drinks, beverages containing fruit juice, soft drinks, milk beverages, butter, mayonnaise, shortening, margarine, various types of salad dressings, bread, noodles, cooked rice, pasta, sauce products, confectioneries, cookies, chocolates, candies, chewing gums, various types of seasonings, and various types of diet products. By incorporating the peptide of the present invention into such a food, the composition of the present invention may be prepared in the form of a food.

The content of the peptide of the present invention in the food of the present invention varies depending on the form of the food. It is typically 0.01% to 80% by mass, preferably 0.1% to 75% by mass, more preferably 1% to 70% by mass, and still more preferably 5% to 70% by mass, on a dry basis. Because the peptide of the present invention has high safety, its content can also be further increased. The daily ingestion amount may be ingested at once or may also be ingested in several portions. It is preferable that the ingestion amount can be controlled so as to achieve the above-described daily ingestion amount per adult.

Ingestion of the peptide of the present invention or a salt thereof having an anti-inflammatory effect or the composition of the present invention containing the same can suppress inflammation and be particularly expected to have the effect of preventing, improving, or treating inflammatory diseases or conditions in which tumor necrosis factor and/or interleukin is involved.

The composition of the present invention can contain various additives used for production of pharmaceutical products, foods, and feeds. There may further coexist various active substances. Examples of such additives and active substances include various oils and fats, crude drugs, amino acids, polyhydric alcohol, naturally-occurring polymers, vitamins, minerals, dietary fibers, surfactants, purified water, excipients, stabilizers, pH modifiers, antioxidants, sweeteners, taste components, acidulants, colorants, and aroma chemicals. The peptide of the present invention can be administered in a mixture or combination with one or a plurality of other active ingredients having an ant-inflammatory activity. Thus, the anti-inflammatory composition of the present invention may comprise other active ingredients having an ant-inflammatory activity, in addition to the peptide of the present invention.

Examples of the various oils and fats include vegetable oils and fats such as soybean oil, safflower oil, and olive oil, and animal oils and fats such as beef tallow and sardine oil.

Examples of the crude drugs include oriental bezoar, rehmanniae radix, lycii fructus, royal jelly, gensing, and Lurong.

Examples of the amino acids include cysteine, leucine, and arginine.

Examples of the polyhydric alcohol include ethylene glycol, polyethylene glycol, propylene glycol, glycerin, and sugar alcohol. Examples of the sugar alcohol include sorbitol, erythritol, xylitol, maltitol, and mannitol.

Examples of the naturally-occurring polymers include gum Arabic, agar, water-soluble corn fiber, gelatin, xanthan gum, casein, gluten or gluten hydrolysates, lecithin, and dextrin.

Examples of the various vitamins include vitamins A, D, and K and riboflavin butyrate, in addition to vitamin C (ascorbic acid), vitamin B family, and vitamin E (tocopherol). The vitamin B family includes various vitamin B complexes such as vitamin B1, vitamin B1 derivatives, vitamin B2, vitamin B6, vitamin B12, biotin, pantothenic acid, nicotinic acid, and folic acid. Vitamin B1 and derivatives thereof include all compounds having physiological activity of vitamin B1, such as thiamine or a salt thereof, thiamine disulfide, fursultiamine or a salt thereof, dicethiamine, bisbutytiamine, bisbentiamine, benfotiamine, thiamine monophosphate disulfide, cycotiamine, octotiamine, and prosultiamine.

Examples of the minerals include calcium, magnesium, zinc, and iron.

Examples of the dietary fibers include gums, mannan, pectin, hemicellulose, lignin, β-glucan, xylan, and arabinoxylan.

Examples of the surfactants include glycerin fatty acid ester, sorbitan fatty acid ester, and sucrose fatty acid ester.

Examples of the excipients include saccharose, glucose, corn starch, calcium phosphate, lactose, dextrin, starch, crystalline cellulose, and cyclodextrin.

Examples of other active ingredients having an anti-inflammatory activity include Morinda citrifolia L-derived ingredient, Cbz-Val-Ala-(OMe)-fluoromethylketone, licorice, glycyrrhitic acid, betulin, ursolic acid, propolis, aloe, acerola, eucalyptus extract, matricaria extract, phellodendron bark, camphor, belladonna, indomethacin, ibuprofen, piroxicam, salicylic acid, diclofenac, ketoprofen, naproxen, and piroxicam.

In addition to the abovementioned, for example, taurine, glutathione, carnitine, creatine, coenzyme Q, α-lipoic acid, glucuronic acid, glucuronolactone, theanine, γ-aminobutyric acid, capsaicin, various organic acids, flavonoids, polyphenols, catechins, xanthine derivative, nondigestible oligosaccharides such as fructo-oligosaccharide, or polyvinylpyrrolidone may be blended as additives. The blending amounts of these additives are each properly determined in accordance with the additive type and the desirable amount to be ingested; however, it typically ranges from 0.01 to 30% by mass, preferably 0.1 to 10% by mass.

Production examples and test examples for the peptide and composition of the present invention will be specifically described with reference to the following Examples. However, the present invention is not intended to be limited to these Examples.

### EXAMPLES

### Production Example 1

### Synthesis of pyroGlu-Gln-Gln

PyroGlu-Gln-Gln was synthesized by a solid-phase method using Model 433A Peptide Synthesizer (from ABI).

Automatic synthesis was carried out in the following manner using 2 g of Boc-Gln-Pam resin as a starting material and employing protected amino acids, Boc-Gln and Boc-Glu (OBzl).

(1) Removal reaction of a Boc group from Boc-Gln-Pam resin

(2) Washing

(3) Activation of Boc-Gln

(4) Addition of activated Boc-Gln to Gln-Pam resin for condensation

(5) Washing

(6) Acetylation of an unreacted N-terminal amino group

(7) Washing

(8) Removal reaction of a Boc group from Boc-Gln-Gln-Pam resin

(9) Washing

(10) Activation of Boc-Glu(OBzl)

(11) Addition of activated Boc-Glu(OBzl) to Gln-Gln-Pam resin for condensation

(12) Washing

(13) Acetylation of an unreacted N-terminal amino group

(14) Washing

(15) Boc-Glu(OBzl)-Gln-Gln-Pam resin

The Boc group was removed by treatment with trifluoroacetic acid-dichloromethane (50:50) for 20 minutes. All steps of washing were each repeated three times using dichloromethane. Condensation was performed by adding the Boc-protected amino acids in amounts 5 times the equivalent of the resin-bound amino group in the presence of DCC and HOBt, followed by reaction for 60 minutes.

The resulting Boc-Glu(OBzl)-Gln-Gln-Pam resin was removed from the peptide synthesizer and transferred to another vessel. Thereto were added 1 mL of thioanisole and 0.5 mL of ethanedithiol per gram of the resin, followed by stirring the mixture at room temperature for 10 minutes. Then, 10 mL of hydrogen fluoride was slowly added under cooling with ice, which was then stirred for 30 minutes, followed by distilling off hydrogen fluoride under reduced pressure. The vessel was filled with 100 mL of cold diethylether, and the resultant was stirred for one minute to precipitate peptide and resin. The resultant was collected by filtration with Polyfron Filter PF060 (from Advantec) and washed with cold diethylether (−40° C.). The peptide was dissolved in about 30 mL of trifluoroacetic acid, which was then added dropwise to 300 mL of cold diethylether provided in advance to again precipitate the peptide. The resultant was collected by filtration with a 3 μm-pore PTFE membrane (from Advantec) and washed with cold diethylether (−40° C.). The peptide was dissolved in 2N acetic acid and then lyophilized. Crude peptide (1.21 g) was obtained from 2.35 g of the protected peptide-Pam-resin. The crude peptide was dissolved in water and subjected to cyclization to pyroglutamic acid at 60° C. for 6 hours, followed by lyophilization.

The resulting crude peptide was purified using HPLC under the following conditions.

Column: Inertsil ODS-3, φ20×250 mm (from GL Sciences)

Mobile phase: Gradient from 0.1% trifluoroacetic acid to 35% acetonitrile in 0.1% trifluoroacetic acid

Flow rate: 10 mL/min

Detector: Ultraviolet spectrophotometer, 210 nm

Temperature: 40° C.

The main peak of the HPLC chromatogram was fractionated, and the amino acid sequence of the fractionation product was analyzed using a peptide sequencer. From 1 g of the crude peptide, 0.88 g of purified pyroGlu-Gln-Gln peptide was obtained.

### Production Example 2

### Synthesis of pyroGlu-Leu

PyroGlu-Leu was synthesized by a liquid-phase method using the Boc method.

(1) Condensation of Boc-pyroGlu and HCl Leu-O^{t}Bu

HCl Leu-O^{t}Bu (390 mg) was introduced into an eggplant-shaped flask, dissolved in 5 mL of DMF, and cooled with ice, to which 0.124 mL of triethylamine was then added. Subsequently, 400 mg of Boc-pyroGlu-OH, 470 mg of HOBt, and 367 mg of WSCD HCl were added thereto, which was then stirred for 12 hours under cooling with ice for condensation reaction. After the end of the reaction, DMF was distilled off under reduced pressure, and the residue was dissolved in ethyl acetate. Then, ethyl acetate was washed with a 5% sodium hydrogen carbonate aqueous solution, a 10% citric acid aqueous solution, water, and saturated saline in that order, and the resultant was dried over anhydrous sodium sulfate. Sodium sulfate was filtered off, and the filtrate was concentrated under reduced pressure. To the resulting residue was added ether-hexane to solidify and collect Boc-pyroGlu-Leu-O^{t}Bu. The yield was 609 mg (88%).

(2) Deprotection

The Boc-pyroGlu-Leu-O^{t}Bu (600 mg) obtained above was introduced into an eggplant-shaped flask, to which 5 mL of trifluoroacetic acid was then added for dissolution, followed by deprotection reaction for 1 hour under cooling with ice. Trifluoroacetic acid was removed using N₂ gas, and the deprotected peptide was solidified by adding ether and then collected by filtration. The resulting solid was dissolved in 4N HCl/dioxane, to which ether was then added for resolidification, followed by collection by filtration. The yield was 220 mg (53%).

### Production Example 3

### Synthesis of pyroGlu-Val

PyroGlu-Val was synthesized in the same manner as that for Production Example 2 using 209.7 mg of HCl H-Val-O^{t}Bu as a starting material. The yield of the condensation reaction was 326.6 mg (85%), and the yield of the deprotected peptide was 205.0 mg (91%).

### Production Example 4

### Synthesis of pyroGlu-Met

PyroGlu-Met was synthesized in the same manner as that for Production Example 2 using 241.8 mg of HCl H-Met-O^{t}Bu as a starting material. The yield of the condensation reaction was 208.3 mg (50%), and the yield of the deprotected peptide was 90.3 mg (60%).

### Production Example 5

### Synthesis of pyroGlu-Phe

PyroGlu-Phe was synthesized in the same manner as that for Production Example 2 using 257.8 mg of HCl H-Phe-O^{t}Bu as a starting material. The yield of the condensation reaction was 242.9 mg (56%), and the yield of the deprotected peptide was 103.1 mg (59%).

### Production Example 6

### Synthesis of pyroGlu-Gln-Gln

PyroGlu-Gln-Gln was synthesized by a liquid-phase method using the Fmoc method.

(1) Synthesis of Fmoc-Gln(Trt)-Gln-O^{t}Bu HCl Gln-O^{t}Bu (1.15 g) was introduced into an eggplant-shaped flask, dissolved in 5 mL of DMF, and cooled with ice, to which 0.74 mL of triethylamine was then added. Subsequently, 2.94 g of Fmoc-Gln(Trt)-OH, 1.3 g of HOBt, and 1.01 g of WSCD HCl were added thereto, which was then stirred for 12 hours under cooling with ice for condensation reaction. After the end of the reaction, DMF was distilled off under reduced pressure, and the residue was dissolved in ethyl acetate. Then, ethyl acetate was washed with a 5% sodium hydrogen carbonate aqueous solution, a 10% citric acid aqueous solution, water, and saturated saline in that order, and the resultant was dried over anhydrous sodium sulfate. Sodium sulfate was filtered off, and the filtrate was concentrated under reduced pressure. To the resulting residue was added ether-hexane to solidify and collect Fmoc-Gln(Trt)-Gln-O^{t}Bu. The yield was 3.51 g (92%).

(2) Removal of Fmoc Group from Fmoc-Gln(Trt)-Gln-O^{t}Bu

Fmoc-Gln(Trt)-Gln-O^{t}Bu (1.12 g) was introduced into an eggplant-shaped flask, to which 7 mL of a 1M NaOH aqueous solution was then added under cooling with ice. Since the mixture developed a white turbidity, methanol was added thereto for dissolution, and the solution was reacted at 0° C. for 2 hours. After neutralization by addition of citric acid, water was added to a white solid resulting from vacuum concentration, which was then stirred to provide a gummy solid. This solid was applied to a silica gel column using chloroform as a solvent, and the desired ingredient was fractionated and solidified using ether. The yield was 590 mg (73%).

(3) Synthesis of Boc-pyroGlu-Gln(Trt)-Gln-O^{t}Bu

H-Gln(Trt)-Gln-O^{t}Bu (580 mg) was introduced into an eggplant-shaped flask, dissolved in 5 mL of DMF, and cooled with ice, to which 156 μL of triethylamine was then added. Subsequently, 232 mg of Boc-pyroGlu-OH, 273 mg of HOBt, and 213 mg of WSCD HCl were added thereto, which was then stirred for 12 hours under cooling with ice for condensation reaction. DMF was distilled off under reduced pressure, and the residue was dissolved in ethyl acetate. Then, ethyl acetate was washed with a 5% sodium hydrogen carbonate aqueous solution, a 10% citric acid aqueous solution, water, and saturated saline in that order, and the resultant was dried over anhydrous sodium sulfate. Sodium sulfate was filtered off, and the filtrate was concentrated under reduced pressure. The resulting residue was depressurized using a vacuum pump to remove the solvent. The yield was 509.3 mg (64%).

(4) Deprotection

Boc-pyroGlu-Gln(Trt)-Gln-O^{t}Bu (760 mg) was introduced into an eggplant-shaped flask, to which 10 mL of trifluoroacetic acid was then added for dissolution, followed by reaction for 4 hour under cooling with ice. Trifluoroacetic acid was removed using N₂ gas, and the deprotected peptide was solidified by adding ether. The sold was collected by centrifugation and suspended by again adding ether. The suspension was centrifuged to collect a solid. This operation was repeated three times to provide a crude peptide. The yield was 445 mg (100%).

(5) Purification of pyroGlu-Gln-Gln

The crude peptide obtained above contained water-insoluble impurities. Thus, the crude peptide was suspended in water, and the filtrate was collected through a filter. Into the filtrate was introduced 2 mL of 1M hydrochloric acid, which was then lyophilized. Ether was added to the lyophilization product to solidify the peptide of the present invention, and the solid was collected and then dried. The final yield was 256 mg (63%).

### Production Example 7

### Synthesis of pyroGlu-Pro-Gln

PyroGlu-Pro-Gln was synthesized by a liquid-phase method using the Fmoc method as in Production Example 6. The yield was 174 mg (49%).

### Production Example 8

### Extraction of pyroGlu-Gln-Gln, pyroGlu-Gln, pyroGlu-Leu, and pyroGlu-Ile from Natural Protein

(1) Ion-exchanged water (9,700 kg), anhydrous citric acid (38 kg), and wheat gluten (1,500 kg) (active gluten, from Weston Foods Limited) were charged into a reaction vessel and warmed at 45° C. Then, 2.2 kg of protease (“Protease M Amano” from Amano Pharmaceutical Co., Ltd.) and 1.1 kg of amylase (“Liquefying Enzyme T” from Hankyu Bioindustry Co., Ltd.) were added thereto for hydrolysis at 45° C. for 5 hours. Subsequently, the liquid was adjusted to a pH of 4.4 to 4.5 using a 25% sodium hydroxide aqueous solution and held in such a state for 7 hours for enzyme treatment.

(2) Subsequently, the liquid was maintained at 80° C. for 20 minutes to deactivate the protease. Thereafter, the liquid was cooled to 65° C., to which 0.5 kg of amylase (“Liquefying Enzyme T” from Hankyu Bioindustry Co., Ltd.) was then added to hydrolyze starch and fiber contained in the wheat gluten, followed by deactivating the amylase by maintaining the liquid at 90° C. for 20 minutes.

(3) Next, the liquid was cooled to 10° C. or lower and then again heated to 55° C. Activated carbon (“Takecoal” from Takeda Pharmaceutical Company Limited) (100 kg) was added thereto, which was then stirred at 55° C. for 30 minutes.

(4) The liquid temperature was adjusted to 45° C., and a filter aid (“Radiolite” from Showa Chemical Industry Co., LTD.) was added. Filtration was carried out using a pressure filtration apparatus to recover 7,000 liters (7 m³) of filtrate.

(5) The filtrate recovered in (4) above was concentrated under reduced pressure, sterilized by heating at 110° C. for 20 seconds using a plate heater, and then cooled to 55° C.

(6) The liquid obtained in (5) above was spray-dried using a spray drier under conditions of a blast temperature of 160° C. and an air exhaust temperature of 80° C. to provide about 1,000 kg of powdered wheat gluten hydrolysates.

(7) Fractions with molecular weights of 1,000 or smaller were fractionated from the powder obtained in (6) above using a gel filtration method, and purification was further carried out using HPLC. By HPLC, portions exhibiting the same retention time under the same conditions were collected based on the synthesized pyroGlu-Gln-Gln, pyroGlu-Gln, pyroGlu-Leu, and pyroGlu-Ile obtained in the same manner as in Production Example 1. As a result, 4.5 kg, 1.6 kg, 0.9 kg, and 0.7 kg of peptides, respectively, were obtained from 800 kg of powdered wheat gluten hydrolysates.

(8) The amino acid sequences of the purified peptides were analyzed using a peptide sequencer. As a result, the peptides were found to have sequences pyroGlu-Gln-Gln, pyroGlu-Gln, pyroGlu-Leu, and pyroGlu-Ile.

### Example 1

### Production of Tablet

The pyroGlu-Leu peptide (84 g) obtained in Production Example 8, 10 g of crystalline cellulose (from Asahi Kasei Corporation), and 5 g of polyvinylpyrrolidone (from BASF) were mixed, to which 3 mL of ethanol was then added, followed by producing granules according to a conventional procedure by a wet method. The resulting granules were dried, to which 1.1 g of magnesium stearate was then added to make a granular powder for tableting. The powder was compressed using a tableting machine to produce 100 tablets each having a weight of 1 g (pyroGlu-Gln content per tablet is 0.84 g.).

### Example 2

### Production of Syrup Preparation

Purified water (400 g) was boiled, to which 750 g of saccharose and 100 g of the pyroGlu-Leu peptide obtained in Production Example 8 were then added under stirring up for dissolution. The solution was then subjected to straining during hot state. To the resultant was added purified water to a total amount of 1,000 mL to produce a syrup preparation (pyroGlu-Leu content per 100 mL of syrup preparation is 10 g.).

### Example 3

### Production of Granular Preparation

PyroGlu-Leu peptide obtained in Production Example 8 (76 g), 13.3 g of lactose (from DMV), 6.7 g of crystalline cellulose (from Asahi Kasei Corporation), and 4 g of polyvinylpyrrolidone (from BASF) were mixed, to which 30 mL of ethanol was then added, followed by producing granules according to a conventional procedure by a wet method. After drying, the granules were sized to provide granular preparations (pyroGlu-Ile content per 10 g of granular preparation is 7.6 g.).

### Example 4

### Production of Liquid Meal

Sodium caseinate (from DMV) (40 g), 160 g of maltodextrin (from Sanwa Cornstarch Co., Ltd.), and 25 g of pyroGlu-Leu peptide obtained in Production Example 8 were added to 750 mL of purified water at about 65° C. for dissolution. Subsequently, 5 g of a vitamin mix and 5 g of a mineral mixed solution comprising sodium, potassium, calcium, magnesium, chlorine, iron, phosphorus, copper, zinc, manganese, and sulfide were added thereto. The mixture was charged into a homomixer (from Tokushu Kika Kogyo Co., Ltd.) and roughly emulsified at about 8,000 rpm for 15 minutes. The resulting emulsion was cooled to about 20° C., to which aroma chemicals were then added, followed by dilution in a measuring cylinder to the final amount of 1,000 mL. A pouch was filled with the emulsion (230 g) and sealed while being purged with nitrogen. The liquid was sterilized at 121° C. for 15 minutes to provide a concentrated liquid meal (pyroGlu-Ile content per 230 g of liquid meal is about 5.8 g.).

### Example 5

### Production of Bread

Wheat flour (bread flour) (150 g) was mixed with 2 g of dry yeast. Separately, 20 g of the pyroGlu-Gln-Gln peptide obtained in Production Example 8, 20 g of sugar, 3 g of salt, and 6 g of skimmed milk powder were dissolved in 70 g of warm water, and one chicken egg was added thereto, which was then thoroughly mixed. The resultant was added to the wheat flour, which was then thoroughly kneaded by hand. Then, about 40 g of butter was added thereto, which was further kneaded to prepare dough for 20 bread rolls. Subsequently, after fermenting the dough, a beaten egg was applied to the surface thereof, which was then baked in an oven at 180° C. for about 15 minutes to produce bread rolls (pyroGlu-Gln-Gln content per bread roll is about 1 g.).

### Example 6

### Production of Meat Sauce for Pasta

One serving of a meat sauce for pasta (150 g) was introduced into a pan and 5 g of the pyroGlu-Gln-Gln peptide obtained in Production Example 8 was simultaneously added thereto, which was then heated to prepare a meat sauce for pasta. A pouch was filled with the resulting sauce, sealed while being purged with nitrogen gas, and sterilized at 121° C. for 15 minutes to provide a meat sauce for pasta containing the pyroGlu-Gln-Gln peptide.

### Example 7

### Production of Japanese Wheat Noodle

A dispersion of 15 g of the pyroGlu-Leu peptide obtained in Production Example 8 and 15 g of salt in 150 g of water was added to 300 g of wheat flour (all-purpose flour), which was then thoroughly kneaded and allowed to stand. Thereafter, the dough was stretched and sliced to a width of about 5 mm to produce Japanese wheat noodles. The noodles were cooked in boiling water for about 10 minutes. As a result, the noodles exhibited good appearance, taste and texture. The Japanese wheat noodles contained about 5 g of pyroGlu-Gln peptide per serving.

### Test Example 1

### Measurement of TACE-Inhibiting Activity

One mg/mL each of samples of pyroglutamyl peptides (pyroGlu-Leu, pyroGlu-Val, pyroGlu-Met, pyroGlu-Phe, pyroGlu-Gln-Gln, and pyroGlu-Pro-Gln) synthesized in Production Examples above were prepared and evaluated for TACE-inhibiting activity as follows.

Each sample (10 μL) was added to 10 μL of 1 μmol/L reactive substrate (TACE Substrate (Mac-PLAQAV-Dpa-RSSSR-NH2) from Biomol. International LP), 10 μL of 10 ng/10 μL enzyme solution (Recombinant Human TACE from R&D Systems), 50 μL of buffer (50 mmol/L Tris-HCl, pH 9.0, 5 μM ZnCl₂, 0.01% Brij35), and 20 μL of distilled water, which was reacted at 37° C. for 20 minutes. Thereto was added 10% trifluoroacetic acid to a final concentration of 1% to stop the reaction. The substrate and the product were separated under the following conditions using reversed-phase high-performance liquid chromatography. The substrate and the product were fluorescently measured at an excitation wavelength of 320 nm and a measuring wavelength of 405 nm for quantification.

(Chromatography Condition)

Solution A: 10% acetonitrile (0.1% TFA)/Solution B: 80% acetonitrile (0.1% TFA)

Gradient: Solution B from 50% to 100%

Column: 5C18 AR-II; 4.6 φ×150

Oven Temperature: 30° C.

Measuring Wavelength: 230 nm

The results are shown in Table 1 below as the ratio of the fluorescence intensity of the product to the fluorescence intensity of the product and the substrate.

TABLE 1

| Control (No Peptide) | 100% |
|---|---|
| pyroGlu-Leu | 61% |
| pyroGlu-Pro-Gln | 66% |
| pyroGlu-Gln-Gln, | 70% |
| pyroGlu-Val | 81% |
| pyroGlu-Met | 83% |
| pyroGlu-Phe | 84% |

### Test Example 2

### Measurement of ICE-Inhibiting Activity

One mg/mL each of samples of pyroglutamyl peptides (pyroGlu-Leu, pyroGlu-Val, pyroGlu-Met, pyroGlu-Phe, pyroGlu-Gln-Gln, and pyroGlu-Pro-Gln) synthesized in Production Examples above were prepared and evaluated for ICE-inhibiting activity as follows.

Each sample (5 μL) was added to 10 μL of 2,000 μmol/L reactive substrate (Caspase-1 substrate (Ac-Trp-Glu-His-Asp-AMC) from Alexis Biochemicals), 5 μL of 10 U/μl enzyme solution (Caspase-1 from Biomol. International LP), 60 μL of buffer (50 mmol/L HEPES, pH 7.4, 100 mM NaCl, 0.1% CHAPS, 1 mM EDTA, 10% glycerol, and 10 mM DTT), and 20 μL of distilled water, which was reacted at 37° C. for 20 minutes. Thereto was added 10% trifluoroacetic acid to a final concentration of 1% to stop the reaction. The substrate and the product were separated under the following conditions using reversed-phase high-performance liquid chromatography. The substrate and the product were fluorescently measured at an excitation wavelength of 380 nm and a measuring wavelength of 460 nm for quantification.

(Chromatography Condition)

Solution A: 10% acetonitrile (0.1% TFA)/Solution B: 80% acetonitrile (0.1% TFA)

Gradient: Solution B from 50% to 100%

Column: 5C18 AR-II; 4.6 φ×150

Oven Temperature: 30° C.

Measuring Wavelength: 230 nm

The results are shown in Table 2 below as the ratio of the fluorescence intensity of the product to the fluorescence intensity of the product and the substrate.

TABLE 2

| Control (No Peptide) | 100% |
|---|---|
| pyroGlu-Leu | 55% |
| pyroGlu-Pro-Gln | 62% |
| pyroGlu-Gln-Gln, | 63% |
| pyroGlu-Val | 74% |
| pyroGlu-Met | 75% |
| pyroGlu-Phe | 72% |

All publications, Patents and Patent Applications cited herein are hereby incorporated as reference in their entirety.

1. A peptide comprising an amino acid sequence represented by the formula:
pyroGlu-(X)n-A or a salt thereof,
wherein X are the same or different and are each independently Gln, Asn, or Pro; A represents Gln, Asn, Leu, Ile, Met, Val, or Phe; and n represents an integer of 0 to 2.

2. The peptide or a salt thereof according to claim 1, wherein X represents Gln or Pro; A is Gln, Leu, Met, Val, or Phe; and n represents 0 or 1.

3. The peptide or a salt thereof according to claim 2, wherein the peptide is selected from the group consisting of pyroGlu-Leu, pyroGlu-Val, pyroGlu-Met, pyroGlu-Phe, pyroGlu-Gln-Gln, and pyroGlu-Pro-Gln.

4. An anti-inflammatory composition comprising at least one peptide or salt thereof according to any one of claims 1 to 3 as an active ingredient.

5. The composition according to claim 4, wherein the composition is for suppressing inflammation by inhibiting a tumor necrosis factor-converting enzyme and/or caspase-1.

6. The composition according to claim 4 or 5, wherein the composition is for preventing, improving, or treating an inflammatory disease or condition in which tumor necrosis factor and/or interleukin is involved.

7. The composition according to any one of claims 4 to 6, wherein the composition is in the form of food.
